(19) United States
(12) Patent Application Publication
(10) Pub. No.: US 2004/0018485 A1
(43) Pub. Date: Jan. 29, 2004

(54) Multiplexed analysis of cells

(76) Inventors: Ilya Ravkin, Palo Alto, CA (US); Simon Goldbard, San Jose, CA (US); Katherine M. Tynan, San Carlos, CA (US); Michael A. Zarowitz, San Carlos, CA (US); Oren E. Beske, Sunnyvale, CA (US)

Correspondence Address: KOLISCH HARTWELL, P.C., 520 S.W. YAMHILL STREET, SUITE 200, PORTLAND, OR, 97204, US

(21) Appl. No.: 10/444,573

(22) Filed: May 23, 2003

Related U.S. Application Data
(63) Continuation-in-part of application No. 10/282,904, filed on Oct. 28, 2002.
(63) Continuation-in-part of application No. 10/120,900, filed on Apr. 10, 2002.
Application No. 10/282,904 is a continuation-in-part of application No. 09/694,077, filed on Oct. 19, 2000, now abandoned.
Application No. 09/694,077 is a continuation-in-part of application No. 09/549,970, filed on Apr. 14, 2000.
(60) Provisional application No. 60/383,091, filed on May 23, 2002.
(60) Provisional application No. 60/413,407, filed on Sep. 24, 2002.
(60) Provisional application No. 60/348,025, filed on Oct. 26, 2001.
(60) Provisional application No. 60/129,664, filed on Apr. 15, 1999.
(60) Provisional application No. 60/170,947, filed on Dec. 15, 1999.
(60) Provisional application No. 60/241,714, filed on Oct. 18, 2000.

(30) Foreign Application Priority Data
Oct. 18, 2001 (WO) PCT/US01/51413
May 16, 2002 (WO) 02/37944

(51) Int. Cl. C12Q 1/00

(52) U.S. Cl. 435/4

(57) ABSTRACT

Systems, including methods, apparatus, compositions, and kits, for multiplexed analysis of different cell populations to measure a set of responses generated by the cell populations upon exposure to a condition in a shared fluid volume, to define a selective effect, if any, of the condition. The invention also provides databases that relate sets of responses measured from multiplexed analysis of cell populations in a plurality of shared fluid volumes to different conditions that generated each set of responses.

### CROSS-REFERENCES TO PRIORITY APPLICATIONS

This application is a continuation-in-part of the following U.S. patent application Ser. No. 10/282,904, filed Oct. 28, 2002; and Ser. No. 10/120,900, filed Apr. 10, 2002. This application also is based upon and claims the benefit under 35 U.S.C. §119(e) of the following U.S. provisional patent applications: Serial No. 60/383,091, filed May 23, 2002; Serial No. 60/413,407, filed Sep. 24, 2002; and Serial No. ______, filed May 22, 2003, titled MULTIPLEXED ANALYSIS OF CELLS, and naming Ilya Ravkin, Simon Goldbard, Katherine M. Tynan, Michael A. Zarowitz, and Oren E. Beske as inventors.

U.S. patent application Ser. No. 10/282,904, in turn, is a continuation-in-part of the following U.S. patent application Ser. No. 09/694,077, filed Oct. 19, 2000; and Ser. No. 10/120,900, filed Apr. 10, 2002. The '904 application also is based upon and claims the benefit under 35 U.S.C. §119(e) of U.S. Provisional Patent Application Serial No. 60/348,025, filed Oct. 26, 2001.

U.S. patent application Ser. No. 09/694,077, in turn, is a continuation-in-part of U.S. patent application Ser. No. 09/549,970, filed Apr. 14, 2000; which, in turn, is based upon and claims the benefit under 35 U.S.C. §119(e) of the following U.S. provisional patent applications: Serial No. 60/129,664, filed Apr. 15, 1999; and Serial No. 60/170,947, filed Dec. 15, 1999. The '077 application also is based upon and claims the benefit under 35 U.S.C. §119(e) of U.S. Provisional Application Serial No. 60/241,714, filed Oct. 18, 2000.

U.S. patent application Ser. No. 10/120,900, in turn, claims priority under 35 U.S.C. §120 of PCT Patent Application Serial No. PCT/US01/51413, filed Oct. 18, 2001, and published as Publication No. WO 02/37944 on May 16, 2002, which, in turn, is based upon and claims the benefit under 35 U.S.C. §119(e) of the following U.S. provisional patent applications: Serial No. 60/241,714, filed Oct. 18, 2000; Serial No. 60/259,416, filed Dec. 28, 2000; Serial No. 60/293,863, filed May 24, 2001; Serial No. 60/299,267, filed Jun. 18, 2001; Serial No. 60/299,810, filed Jun. 20, 2001; Serial No. 60/307,649, filed Jul. 24, 2001; Serial No. 60/307,650, filed Jul. 24, 2001; Serial No. 60/310,540, filed Aug. 6, 2001; Serial No. 60/317,409, filed Sep. 4, 2001; Serial No. 60/318,156, filed Sep. 7, 2001; and Serial No. 60/328,614, filed Oct. 10, 2001.

The above-identified U.S., PCT, and provisional patent applications are all incorporated herein by reference in their entirety for all purposes.

### CROSS-REFERENCES TO RELATED APPLICATIONS

This application incorporates by reference in their entirety for all purposes the following U.S. patent application Ser. No. 09/549,970, filed Apr. 14, 2000; Ser. No. 10/119,814, filed Apr. 9, 2002; Ser. No. 10/186,219, filed Jun. 27, 2002; Ser. No. 10/238,914, filed Sep. 9, 2002; Serial No. 10/273,605, filed Oct. 18, 2002; Ser. No. 10/282,940, filed Oct. 28, 2002; Ser. No. 10/382,796, filed Mar. 5, 2003; Ser. No. 10/382,797, filed Mar. 5, 2003; Ser. No. 10/382,818, filed Mar. 5, 2003; Ser. No. 10/407,630, filed Apr. 4, 2003; and Ser. No. ______ , filed May 23, 2003, titled ASSAYS WITH CODED SENSOR PARTICLES TO SENSE ASSAY CONDITIONS, and naming Oren E. Beske and Simon Goldbard as inventors.

This application incorporates by reference in their entirety for all purposes the following U.S. provisional patent applications: Serial No. 60/383,092, filed May 23, 2002; Serial No. 60/426,633, filed Nov. 14, 2002; Serial No. 60/469,508, filed May 8, 2003; and Serial No. ______ , filed May 22, 2003, titled MULTIPLEXED ANALYSIS OF CELLS, naming Ilya Ravkin, Simon Goldbard, Katherine M. Tynan, Michael A. Zarowitz, and Oren E. Beske as inventors.

### FIELD OF THE INVENTION

The invention relates to multiplexed analysis of cell populations. More particularly, the invention relates to multiplexed analysis of cell populations to measure a set of responses generated by the cell populations upon exposure to a condition in a shared fluid volume, to define a selective effect, if any, of the condition.

### BACKGROUND

High-throughput screens may test the biological activity of a library of compounds on cells. Each compound may be analyzed individually or as part of a pool of library compounds for its ability to elicit a response from the cells. The measurement of a response from cells may provide a more biologically relevant indication of the bioactivity of a compound than a noncellular biochemical assay. Accordingly, the use of cells may identify viable drug candidates with improved efficiency.

A high-throughput screen of a library of compounds may be performed stepwise. In a primary segment of the screen, the compounds may be tested for their ability to elicit a response from cells of a desired target population. Positive compounds or “hits” from this primary segment then may be further characterized in a secondary segment of the screen. The secondary segment may test for selectivity (specificity) of the positive compounds on a panel of specificity targets, which may be cell populations that are engineered to differ from the desired target population. The secondary segment may determine information about selective activity of the positive compounds on the desired target population relative to the panel of specificity targets.

Cross-reactivity information then may be used to develop structure-activity relationships (SAR). In particular, chemists may search for common structural features among the positive compounds, in an attempt to identify a common structure that confers sufficient potency and selectivity in eliciting a response from the desired target population. SAR information may be very valuable to the chemists as it may be used alone, or in combination with other SAR information gained from other screens, to guide derivatization of one or more of the positive compounds.

Potency and selectivity analyses performed stepwise, as described above, may have a number of disadvantages. For example, selectivity information may be obtained only on strongly positive compounds identified in the primary segment of the screen. However, weaker compounds that exhibit less activity in the primary segment also may provide valuable information in a selectivity analysis. For example, the weaker compounds may act more specifically on the desired target than the strongly positive compounds, offering structural insight into specificity. Alternatively, the weaker compounds may show high cross-reactivity, offering additional insight into structures that lack the necessary specificity toward the desired target. Therefore, methods are needed that allow potency and selectivity analyses to be combined during analysis, providing selectivity information about each member tested in a library screen.

### SUMMARY

The invention provides systems, including methods, apparatus, compositions, and kits, for multiplexed analysis of different cell populations to measure a set of responses generated by the cell populations upon exposure to a condition in a shared fluid volume, to define a selective effect, if any, of the condition. The invention also provides databases that relate sets of responses measured from multiplexed analysis of cell populations in a plurality of shared fluid volumes to different conditions that elicited each set of responses.

### BRIEF DESCRIPTION OF THE DRAWINGS

FIG. 1 is a flowchart of a method of screening a set of conditions for the ability to elicit selective responses from cell populations in a nonpositional array, in accordance with aspects of the invention.

FIG. 2 is a flowchart of selected portions of a method of screening a set of conditions for the ability to elicit selective responses from cell populations in a positional array, in accordance with aspects of the invention.

FIG. 3 is a three-dimensional plot showing a graphical method of presenting data from a selectivity analysis of the responses of desired targets, a selectivity panel, and a toxicology panel to exposure to different conditions in different wells, in accordance with aspects of the invention.

FIG. 4 is a graph of comparative results obtained using cells connected to microplate wells or to coded carriers for assay of activation of the diuretic hormone receptor with a range of concentrations of diuretic hormone, in accordance with aspects of the invention.

FIG. 5 is a graph of activity measurements obtained using cell populations connected to coded carriers and expressing a set of receptors and controls, including G protein-coupled receptors and a nuclear hormone receptor, but without exposure to diuretic hormone, in accordance with aspects of the invention.

FIG. 6 is a graph of activity measurements obtained as in FIG. 5, but in the presence of added diuretic hormone, in accordance with aspects of the invention.

FIG. 7 is a graph of activity measurements obtained as in FIG. 5, but with the addition of a compound (“Compound X”) that may act as an agonist of an endogenous G-Protein Coupled Receptor, in accordance with aspects of the invention.

FIG. 8 is a three-dimensional plot showing hypothetical results from multiplexed analysis using coded carriers to identify relationships between receptors and/or compounds, and differences in potency and specificity of compounds, in accordance with aspects of the invention.

FIG. 9 is a graph of BrdU incorporation produced with exposure of three different cell types to a range of topotecan concentrations and analyzed in multiplex with coded carriers according to FIG. 1, in accordance with aspects of the invention.

FIG. 10 is a graph produced according to FIG. 9 but with exposure to irinotecan rather than topotecan, in accordance with aspects of the invention.

FIG. 11 is a view of results obtained from a screen of known and candidate cytotoxic agents for effects on BrdU incorporation in two cell types, according to the method of FIG. 1.

FIG. 12 is another view of the results of FIG. 11, with selective effects on the cell types represented by distinct indicia, in accordance with aspects of the invention.

### DETAILED DESCRIPTION

The invention provides systems, including methods, apparatus, compositions, and kits, for defining a selective effect, if any, of a condition on responses elicited from two or more cell populations exposed to the condition in a shared fluid volume. The condition may be physical, chemical, and/or biological. In addition, the shared fluid volume may be defined by any suitable vessel, such as a microplate well.

The cell populations may be exposed separately to two or more different conditions in corresponding shared fluid volumes to define any selectivity of each condition on eliciting one or more of the responses generated by the cell populations. For example, the conditions may be provided by exposure to a library of test compounds or materials, such as nucleic acids, proteins, peptides, small molecules, enzymes, antibodies, lipids, mixtures, extracts, viruses, and/or cells, among others. Each member of the library or may be tested for the member's ability to elicit a response from each of the cell populations, to define a set of responses. The responses may be compared to define a selective effect, if any, of the library member on one or more of the responses. Accordingly, a library member (or condition) with a desired selectivity may be identified.

Databases may be created that relate measured responses from cell populations exposed to each condition. For example, analyses may be performed in microplate wells, with each well having a different condition. A corresponding database may relate each well, and thus each different condition, to a set of responses measured therein, or results from wells exposed to the same condition may be averaged.

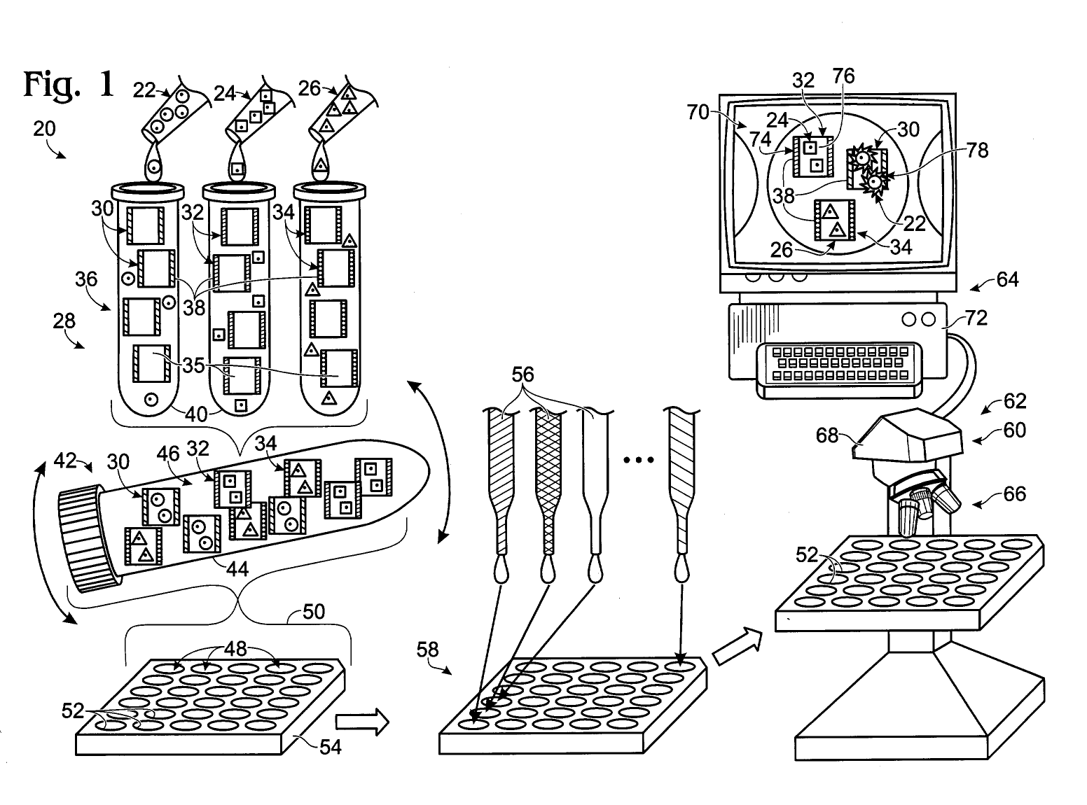

FIG. 1 shows a method 20 of screening a set of conditions for the ability of each condition to elicit a selective response from cell populations in a nonpositional array. Method 20 may be conducted with two or more cell populations 22, 24, 26. Any suitable cell populations may be selected for use in the method, as described in more detail below in Section I.

Method 20 may include a step of placing different cell populations 22, 24, 26 in a shared fluid volume, shown at 28. The cell populations may be in a segregated configuration, that is, disposed at separate positions within an examination site, such as a microplate well 30, without substantial intermixing of individual cells from different populations. Maintaining cells in a segregated configuration may enable each cell population to be identified according to a linked code.

The step of placing 28 may include connecting each cell population to a different class 30, 32, 34 of one or more coded carriers 35, shown at 36. Each coded carrier may include a distinguishable code 38 that identifies the cell population connected to the carrier. The step of connecting 36 may be conducted with each class of coded carrier and each corresponding cell population in fluid isolation, such as in vessels 40, to reduce connection of cells from other cell populations to a noncorresponding class of coded carriers.

The step of placing 28 may include mixing the different classes of coded carriers and their connected cell populations, shown at 42. Mixing may be conducted in a vessel, such as a screw-cap tube 44, or other suitable container. Mixing may include inversion, vortex action, and/or agitation, among others, which may provide a nonpositional mixture 46 in which the different classes of carriers 30, 32, 34 are randomly or arbitrarily distributed relative to one another. Alternatively, different classes of carriers and their connected cell populations may be mixed at an examination site.

The step of placing may include dispensing array portions of nonpositional mixture 46 to examination sites 48, shown at 50. Each array portion may represent each cell population 22, 24, 26 and thus each class of coded carrier 30, 32, 34. Each examination site may be a surface or a vessel, such as a microplate well 52 included in microplate 54. Further aspects of placing cell populations in a shared fluid volume are described below in Section II.

Method 20 may include exposing cell populations 22, 24, 26 (or subpopulations thereof) to different conditions 56, shown at 58. Exposure to the conditions may be performed by addition of a chemical and/or biological agent, such as a test compound, to each well, and/or by treatment with a physical condition. Each set of cell populations may be exposed to each condition for any suitable amount of time. Further aspects conditions and exposure to conditions are included in Section III below.

Method 20 may include measuring responses of the different cell populations elicited by exposure to each condition 56, shown at 60. Measuring responses may be conducted with the cell populations in microplate wells 52. Responses may be measured with an image capture device 62 and an image analysis device 64. The image capture device may include optics 66, such as those provided by a microscope 68 and a sensor, such as a CCD array or a digital camera. The image capture device may image a field of view from the examination site to create at least one image 70 of the coded carriers and the cell populations. In some embodiments, the image may be two or more images produced from the same field of view with different optics, for example, with different filters, or with a different light source or detector configuration.

Image analysis device 64 may be configured to extract information from the image and to further process the extracted information. Accordingly, image analysis device 64 may include a digital computing device 72 with a processor or controller to perform data manipulation, and a memory to store instructions and data, among others. The instructions may direct the processing and extraction of information from the image.

Image 70 may include identifying information 74 for each cell population. The identifying information may include code images 38 that identify cell populations connected to the different carrier classes 30, 32, 34. The identifying information also may include a cell-association area 76 defined by each carrier. The position of the cell-association area may be defined, for example, relative to the perimeter of each carrier, relative to the coding regions, and/or the like. The cells belonging to each cell population may be inferred based on proximity to the position of the cell-association area defined by each carrier.

Image 70 also may include response information 78 for each cell population. The response information may include a signal sensed for each cell population. The signal may be derived from the image by image analysis device 64 The signal may be determined, for example, by defining an area of the image occupied by each cell population, masking a portion of the image substantially complementary to the area, and collecting the signal selectively from the unmasked portion corresponding to the area. Response signals may be compared to control values or to expected values, among others, to define each response. Furthermore, responses may be compared with one another to define a selectivity of the condition for eliciting a subset of the responses. Further aspects of measuring responses and defining the selectivity of a condition are described below in Sections IV and V.

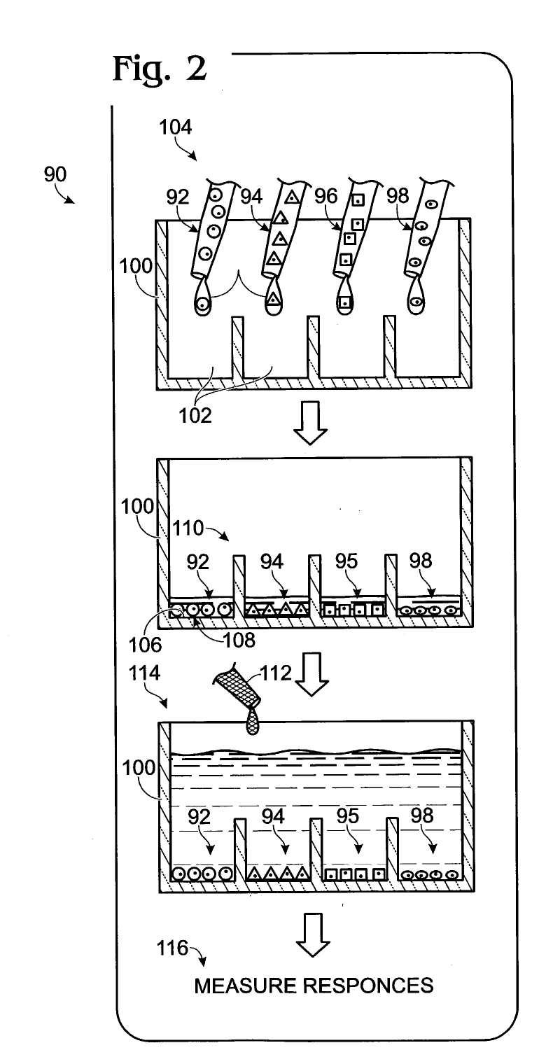

FIG. 2 shows selected portions of a method 90 of screening a set of conditions for the ability of each condition to elicit a selective response from cell populations in a positional array. Positional method 90 may differ from method 20 above in using relative or absolute positions of cell populations 92, 94, 96, 98 within a subdivided microplate well 100 to identify each cell population.

Method 90 may include placing different cell populations 92-98 at predefined positions within well 100. Each cell population may be placed in a different sub-well 102, with the cell populations in fluid isolation from one another, shown at 104. Alternatively, the cell populations may be placed while the sub-wells are in fluid communication, for example, with each cell population directed to a desired sub-well by gravity. Accordingly, each cell population may be connected to particles having or lacking codes or may be connected to each sub-well, adjacent a surface 106 of each sub-well, shown at 108. In some embodiments, the cell populations may be connected to known positions of an insert placed in the well, such as a disk, or may be connected to predefined positions of a well that is not subdivided. In any case, a positional array 110 of cell populations may be created.

Positional array 110 may be exposed to a condition 112, shown at 114. Different arrays within a microplate, that is, within different wells, may be exposed to different conditions. In some embodiments, exposure to a condition may be effected by adjusting the volume in the well to achieve fluid communication between the cell populations in the well. However, the cell populations may remain at least substantially in a segregated configuration, as described above, during this and a subsequent step.

A response generated by each cell population with exposure to condition 112 maybe measured, shown at 116. Each response may be measured in situ from the cell populations, that is, within the microplate well. Measuring a response may be conducted at least partially as described above for nonpositional method 20. For example, at least one image of the cell populations may be created. The image may include positional identifying information about the cell populations and response information corresponding to the response elicited by the condition from each cell population. Accordingly, an image capture device and an image analysis device may be suitable to measure the responses.

Measuring a response may include identifying each cell population based its associated code and/or position. Therefore, a plurality of responses elicited by each condition may be measured from a single exposure of the condition to the cell populations. This approach combines primary and secondary screens of test compounds. As a result, libraries of test compounds may be screened more effectively to yield increased amounts of information about the biological activity of the compounds, speeding identification of viable drug candidates and effective derivatization of these candidates.

Further aspects of the invention are described in the following sections: (I) cell populations, (II) arrays of cell populations, (III) exposure to conditions, (IV) measurement of responses, (V) selectivity of a condition, (VI) databases and graphical display of data, and (VII) examples.

### I. CELL POPULATIONS

Multiplexed assays may be performed with cell populations. Cell populations generally include any set of one or more cells. The set may include a single type of cells or a mixture of different cell types. Different cell types and populations, as used herein, include cells that are different from one another in one more aspects. The cells may differ biochemically, genetically, and/or phenotypically. Different cell types/populations may have different origins (such as cells from different species, tissues, genetic backgrounds, growth or treatment regimens, etc.) or may be substantially similar cell populations that are engineered to be different (see below). Cells in a cell population may be from any suitable species, and of any suitable type or kind. The cells may be cultured, primary, transformed, included in a tissue, clonal, etc. In some embodiments, the cells of some or all of the cell populations are isolated, that is, separated from their natural proximity to other cells. Exemplary isolated cells are obtained from a tissue by disruption of the tissue and at least partial purification of one or more cell types from the tissue.

Cell populations may include primary cells or cells that have been transformed and/or immortalized by exposure to chemicals, nucleic acids, viruses, radiation, repeated passage, and/or the like. Cells may be grown in culture for one, two, or more divisions or may be obtained directly from an organism, for example, as a blood sample, a biopsy, an explant, etc., or may be obtained from a natural source (such as soil, a body of water, etc.).

Similar cells or cell populations may be engineered to be different by introduction of transfection materials, such as nucleic acids. The transfection materials may be introduced by transfection (such as infection, lipofection, endocytosis of particles, injection, etc.). The transfection materials may be stably integrated into the genomes of one or more cells of a cell population, and/or the materials may be extrachromosomal in one or more cells. Extrachromosomal nucleic acids may be replicating, for example, as episomes, or may be nonreplicating. Nucleic acids may encode and direct expression of a protein. Examples of encoded proteins include cell-surface receptors, such as ion channel-linked receptors (neurotransmitter receptors), G protein-coupled receptors (GPCRs), enzyme-linked receptors (such as tyrosine kinases, serine-threonine kinases, phosphatases, etc.), and/or the like. Other receptors may include nuclear receptors, such as receptors for steroid hormone, thyroid hormones, vitamin D, retinoids, and/or the like. Alternatively, or in addition, the encoded proteins may include transcription factors (for example, AP-1, SP-1, NF-κB, etc.), cell cycle regulators (such as cyclins and cyclin dependent kinases), proteins involved in signaling cascades (protein kinase C, Janus kinases, ERK kinases, MAP kinases, protein kinase A, PI3-kinase, ras, etc.), cytoskeletal proteins (such as actins, tubulins, intermediate filament proteins, and/or associated proteins), transporters, ion channels, extracellular matrix proteins, enzymes, and/or so on.

A cell population may be engineered to include one or more reporter genes in one or more cells of the population. Reporter genes may include regulatory sequences that provide a regulated transcriptional response. Such regulatory sequences may include promoters, enhancers, and/or target elements that respond to a particular signaling pathway, transcription factor, or set of transcription factors. For example, regulatory sequences may include nuclear receptor response elements, cyclic AMP response elements, NFAT response elements, interferon response elements, and/or the like.

Reporter genes express reporter RNAs and/or reporter proteins. Expression level of the reporter RNAs and/or proteins may be measured to provide a response signal generated by the cell population. Reporter proteins may include enzymes (for example, beta-galactosidase, chloramphenicol acetyltransferase, glucuronidase, luciferase, and so on), and/or optically detectable proteins (such as green fluorescent protein or its yellow, red, orange, or blue derivatives and so on).

A cell population used to generate a response includes living cells. The living cells may constitute any portion of the cell population including a minor portion or a substantial portion. By contrast, dead or living cells in a cell population may be suitable for measuring some interactions, such as binding. Living cells may be killed after they generate a response, for example, by fixation, to facilitate measuring the response. Suitable fixatives may include an organic solvent, paraformaldehyde, glutaraldehyde, picric acid, and/or the like, or the cells may be left unfixed, whether alive or dead.

Exemplary cells and cell populations, and methods of engineering cell populations for transfection, are described further in the patent applications listed above under Cross-References, which are incorporated herein by reference, particularly the following U.S. patent application Ser. No. 10/120,900, filed Apr. 10, 2002; and Ser. No. 10/382,818, filed Mar. 5, 2003.

### II. ARRAYS OF CELL POPULATIONS

Multiplexed assays may be performed with an array of cell populations placed at a plurality of examination sites. The array generally comprises any set of two or more cell populations in fluid communication within a shared fluid volume.

Each cell population may be segregated from other cell populations of the array within the fluid volume. Segregated cell populations maintain the cells substantially grouped in one or more groups, without substantial intermingling of cells from different populations. Segregated cell populations may occupy substantially nonoverlapping regions within a shared fluid volume at any given time during an assay. Accordingly, segregated cell populations may have fixed relative or absolute positions at the examination site, such as within a fixed array. Alternatively, segregated cell populations may be mobile relative to one another, such as with a nonpositional array formed by coded carriers, as described in relation to FIG. 1 above.

The shared fluid volume may be defined by a container or vessel, such as a microplate well, a microcentrifuge tube, or a region of a generally planar surface, among others. The array may be a positional array, in which each cell population has a fixed position. Alternatively, or in addition, the array may be a nonpositional, or arbitrarily positioned array, in which at least some or all cell populations of the array have an arbitrary position relative to each other.

A positional array may be formed by positioning cell populations relative to each other and/or relative to a fixed structure. Cell populations positioned relative to each other are identifiable based at least partially on these relative (or absolute) positions. For example, cell populations may be distributed in a spaced array on a substrate, using an asymmetrical arrangement for the purpose of orientation. Alternatively, or in addition, cell populations may be distributed in a spaced array on a substrate having a landmark structure, such as a recess, a protrusion, a marking, and/or so on. In either case, an individual cell population may be identified based on the position of the cell population relative to other populations and/or relative to the landmark structure. In some embodiments, the array may be defined by sub-wells of a subdivided microplate plate well, as described above in relation to FIG. 2.

A nonpositional array may be formed by placing cell populations together in a shared fluid volume after connection of the cell populations to coded carriers. Each cell population may be connected separately to a different class of coded carriers having a different code (or codes). Each cell population then may be identifiable independent of its position within a shared fluid volume and independent of its position relative to other cell populations, based on reading a linked code. Accordingly, an array of identifiable cell populations may be formed by mixing distinct classes of carriers and their connected cell populations. In some embodiments, cell populations may be distributed in a partially positional array in which each target is included in a nonpositional array that has a position within a higher order array, for example, formed by the wells of a microplate. In such a partially positional array, a cell population may be identified by an associated code in combination with a position within the higher order array.

An array of cell populations may be formed from cells that have a common characteristic and/or that differ in any suitable properties. For example, an array of cell populations may include target cell populations that differ to generate different responses to a condition to which the populations are exposed. Such cell population targets may include distinct cell types, such as fibroblasts, myoblasts, neuroblasts, keratinocytes, chrondroblasts, and/or so on. Alternatively, such targets may include a set of generally clonal cell populations that differ according to treatment, such as introduction of a foreign nucleic acid, hormone treatment, growth condition, passage number, viral infection, chemical treatment, radiation exposure, and/or the like. Distinct foreign nucleic acid(s) may result in expression of distinct proteins, may inhibit expression of distinct proteins, and/or may include reporter genes that report different activities in the cells. For example, a target array may include a set of target cell populations that express distinct receptor proteins, but have a common reporter gene that responds to these distinct receptor proteins. Alternatively, or in addition, the target array may include target cell populations that carry distinct foreign reporter genes that are activated by distinct signal transduction pathways. Additional exemplary reporter genes, and expressed proteins provided by foreign nucleic acids, are described above in Section I.

Further aspects of arrays formed using coded carriers or subdivided microplate wells are described in the patent applications identified above under Cross-References, which are incorporated herein by reference, particularly the following U.S. patent application Ser. No. 09/694,077, filed Oct. 19, 2000; Ser. No. 10/120,900, filed Apr. 10, 2002; Ser. No. 10/273,605, filed Oct. 18, 2002; Ser. No. 10/282,940, filed Oct. 28, 2002; and Ser. No. 10/382,818, filed Mar. 5, 2003.

### III. EXPOSURE TO CONDITIONS

An array of cell populations may be exposed to a condition at an examination site to test the effect or activity of the condition on each cell population. The condition may be a physical condition of the examination site, a chemical condition of the site, and/or a biological condition of the site. Exposure may be for any suitable time. The exposure may be continuous, transient, periodic, sporadic, etc.

Physical conditions include any physical state of the examination site. The physical state may be the temperature or pressure of the site, or an amount or quality of light (electromagnetic radiation) at the site. Alternatively, or in addition, the physical state may relate to an electric field, magnetic field, and/or particle radiation at the site, among others.

Chemical conditions include any chemical aspect of the shared fluid volume in which the cell populations are disposed. The chemical aspect may relate to presence or concentration of a test compound or material, pH, ionic strength, and/or fluid composition, among others.

Biological conditions include any biological aspect of the shared fluid volume in which are cell populations are disposed. The biological aspects may include presence/absence/concentration/activity/type of cells, viruses, vesicles, organelles, biological extracts, and/or biological mixtures, among others.

Multiplexed assays may screen a library of conditions to test the activity of each library member on a set of cell populations. A library generally comprises a collection of two or more different members. These members may be chemical modulators (or candidate modulators) in the form of molecules, ligands, compounds, transfection materials, receptors, antibodies, and/or cells (phages, viruses, whole cells, tissues, and/or cell extracts), among others, related by any suitable or desired common characteristic. This common characteristic may be “type.” Thus, the library may comprise a collection of two or more compounds, two or more different cells, two or more different antibodies, two or more different nucleic acids, two or more different ligands, two or more different receptors, or two or more different phages or whole cell populations distinguished by expressing different proteins, among others. This common characteristic also may be “function.” Thus, the library may comprise a collection of two or more binding partners (e.g., ligands and/or receptors), agonists, or antagonists, among others, independent of type.

Library members may be produced and/or otherwise generated or collected by any suitable mechanism, including chemical synthesis in vitro, enzymatic synthesis in vitro, and/or biosynthesis in a cell or organism. Chemically and/or enzymatically synthesized libraries may include libraries of compounds, such as synthetic oligonucleotides (DNA, RNA, peptide nucleic acids, and/or mixtures or modified derivatives thereof), small molecules (about 100 Da to 10 KDa), peptides, carbohydrates, lipids, and/or so on. Such chemically and/or enzymatically synthesized libraries may be formed by directed synthesis of individual library members, combinatorial synthesis of sets of library members, and/or random synthetic approaches. Library members produced by biosynthesis may include libraries of plasmids, complementary DNAs, genomic DNAs, RNAs, viruses, phages, cells, proteins, peptides, carbohydrates, lipids, extracellular matrices, cell lysates, cell mixtures, and/or materials secreted from cells, among others. Library members may be contact arrays of cell populations singly or as groups/pools of two or more members.

Further aspects of conditions (also termed modulators) and libraries of modulators are described in the patent applications listed above under Cross-References, which are incorporated herein by reference, particularly U.S. patent application Ser. No. 10/120,900, filed Apr. 10, 2002.

### IV. MEASUREMENT OF RESPONSES

The effect of exposure to a condition, if any, may be measured as a response to the condition generated by each cell population of an array in a multiplexed assay. The response generally includes any change in, or effect on, the cell population, or lack of change or effect, produced by the condition. Accordingly, measuring the response may include determining whether the condition has any effect on the cell population, and, if so, how much of an effect. Thus, the magnitude of the effect may define a potency of the condition for generating the response.

The response may be measured by sensing a signal for a parameter from the cell population and comparing the signal to a value, to define their relationship. The value may be a predicted (expected) value or a measured value. The predicted value may correspond to a signal value expected for at least substantially no effect of the condition or to a signal value expected by an effect of a particular strength. Accordingly, the predicted value may be defined without performing an experimental measurement. By contrast, the measured value may be a control value produced experimentally, for example, by a negative and/or positive control for a response. The control value may be sensed internally, that is, from a control cell population within the array, or externally, that is from a separate cell population at a separate examination site. The control value may be detected at any suitable time relative to a signal from each experimental cell population.

The response may relate to any measurable parameter (characteristic) of each cell population. The parameter may reflect cytotoxicity, cell proliferation, apoptosis, receptor activity, transformation, immortalization, activity of a signal transduction pathway, cell differentiation, cell movement, cell morphology, gene expression, intracellular trafficking, secretion, endocytosis, channel activity, electrical activity, cell cycle regulation, and/or protein processing, among others.

Cytotoxicity and/or proliferation may be measured by any suitable method using any suitable reagents. Exemplary cytotoxicity assays include the LIVE/DEAD assay from Molecular Probes, staining with 3-(4,5-dimethylthiazoyl-2-yl) 2,5 diphenyltetrazolium bromide (MTT), counting cells, and measuring nuclear morphology, among others. Exemplary proliferation assays may include, but are not limited to, BrdU labeling or measurement of a mitotic index. Further aspects of measuring cytotoxicity and/or proliferation in multiplexed assays are included below in Section VII, particularly Example 5.

Apoptosis may be measured by any suitable method using any suitable reagents. Exemplary apoptosis assays include, but are not limited to, a TUNEL assay, a cell-based caspase assay, and staining for Annexin V.

Cells characteristics or parameters may include localization/movement, modification, morphology, structure, conformation, and/or activity of any cellular component, complex, structure, organelle, and/or whole cells, among others. Examples of levels of cellular components may include levels of total RNA, tRNAs, specific mRNAs, and/or hnRNAs, among others; levels of proteins, peptides, glycoproteins, proteoglycans, and/or reporter proteins (such as beta-galactosidase, luciferase, green fluorescent protein, chloramphenicol acetyltransferase, and/or the like), among others; levels of lipids, such as specific phosphoinositides and forms of cholesterol; and/or the like. Examples of localization may include localization of a component or complex to a cellular organelle or region, such as the nucleus, cytoplasm, Golgi apparatus, lysosomes, nuclear membrane, endoplasmic reticulum, endosomes, cell membranes, cell-surface, extracellular matrix, etc. Accordingly, changes in localization may include transfer of a component between any two or more of these structures or movement of one of these structure itself, such as by nuclear translocation. Examples of modification may include phosphorylation, acetylation, methylation, glycosylation, amidation, gamma-carboxylation, ubiquitination, farnesylation, and/or the like. Conformation or structure may include primary, secondary, tertiary, or quaternary structural aspects. Exemplary changes in conformation or structure may be mediated by cleavage enzymes, ligases, isomerases, epimerases, gyrases, topoisomerases, molecular interactions, etc. Examples of morphologies may include shape of cells, organelles, and membranes, among others. Examples of activities include enzyme activities, electrical activities (such as ion currents or membrane voltages), and/or the like.

Cell characteristics as responses may be measured in multiplexed assays according to the nature of the characteristics, engineering of the cell populations (such as to express GFP), and the nature of the reagents used to perform the assays (such as dyes). Exemplary methods of detecting cell characteristics may include spectroscopic methods (such as absorbance, fluorescence, reflectance, scattering, etc.), surface plasmon resonance, magnetic methods, or electrical methods, among others. Responses may be represented by a single measurement from each cell population, by a sum of individual measurements from a cell population (such as from individual cells in the cell population), and/or by subcellular measurements from a cell population, among others. In some embodiments, responses may be measured from digital images of cell populations.

Measuring a response may include identifying each cell population for which the response is being measured. Each cell population may be identified based on a code and/or a position, as described above in Section II. Identification of cell populations may be carried out before, during, and/or after measuring a signal from each cell population.

Further aspects of measuring responses (as cell characteristics), exemplary cell characteristics, and identifying cell populations for which cell characteristics are measured, are described in more detail in the patent applications identified above under Cross-References, which are incorporated herein by reference, particularly the following U.S. patent application Ser. No. 10/120,900, filed Apr. 10, 2002; and Ser. No. 10/282,904, filed Oct. 28, 2002.

### V. SELECTIVITY OF A CONDITION

Measured responses generated with exposure of cell populations to a condition in a multiplexed assay may be further processed to define a selectivity, if any, of the condition in the assay. A selectivity may include any ability of the condition to produce a selective effect on less than all of the cell populations. The terms selectivity and specificity, as used herein, are intended to have the same meaning.

The selective effect may be defined by comparing or distinguishing the responses. Comparing or distinguishing may be a comparison of detected signals relative to one another, to define a signal (or signals) and thus one or more responses that are greater or less than the other responses. The responses may correspond to the same parameter or different parameters. Accordingly, the responses may be compared directly or may be adjusted, for example, weighted or scaled, among others, before comparison to allow comparison of different types of measurements corresponding to different measured parameters.

### VI. DATABASES AND GRAPHICAL DISPLAY OF DATA

Multiplexed analysis according to the invention may be used to determine structure-activity relationships and/or other information for a library of compounds in one or more screens. This analysis may generate data on combinations of targets in a novel way, resulting in unique databases of information. In particular, multiplexed technology may be used to collapse aspects of both the primary and secondary screens. As a result, multiplexed technology may collect information such as specificity from an entire library rather than merely from those members of the library that are identified as “hits” in the primary screen. Therefore, multiplexed technology may generate a comprehensive database suitable not only for SAR determination but also to guide rational design of future screens. These unique databases may not only be more comprehensive than databases compiled using nonmultiplexed technology, but they also may be used in ways that these other databases cannot.

Databases generated according to the invention may be collected, stored, manipulated, and/or displayed using any suitable mechanism. Typically, elements of the databases will be treated as ordered arrays, that is, as collections of identifying indicia and results. In some embodiments, the results may be represented as response indicia that indicate potency and/or selectivity from one or more multiplexed assays. Exemplary databases and a graphical method for displaying and analyzing results are described below in Examples 1 and 5.

### VII. EXAMPLES

The following examples describe selected aspects and embodiments of the invention, including methods for multiplexed analysis of responses generated by exposure of cell arrays to conditions, and results obtained using these methods. These examples are included for illustration and are not intended to limit or define the entire scope of the invention.

### Example 1.

Graphical Display of Multiplexed Analyses

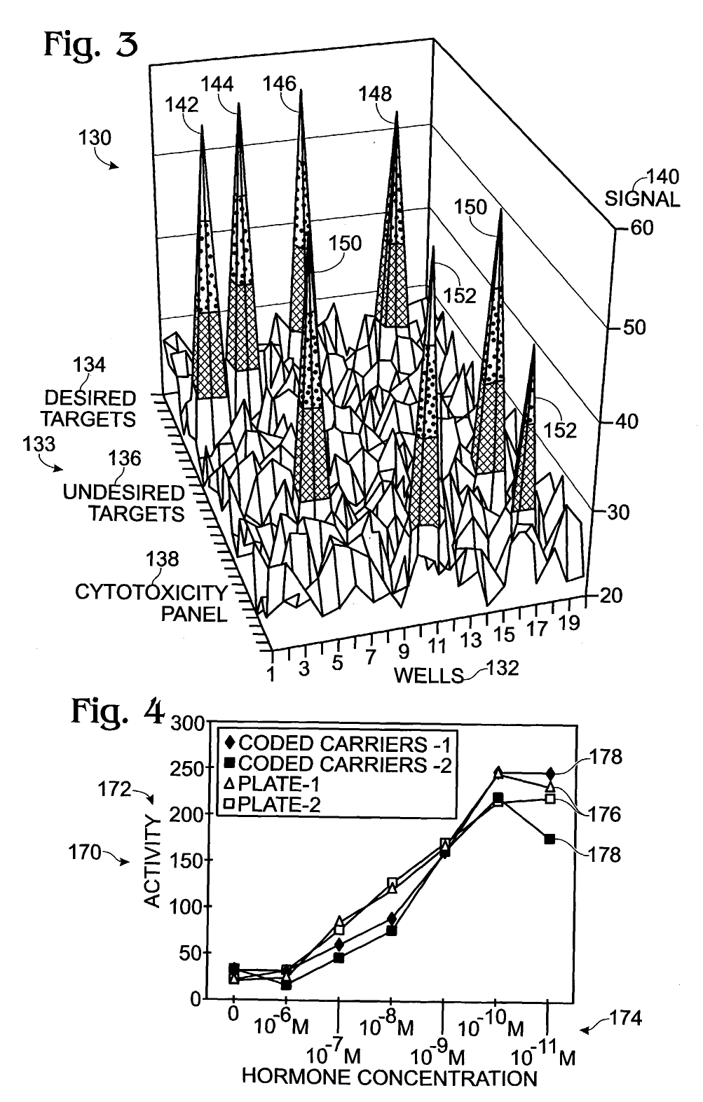

This example describes results that may be obtained from a selectivity analysis of the responses of desired targets, a selectivity panel, and a toxicology panel produced by exposure to different conditions in different microplate wells. This example also illustrates a graphical method of displaying such results; see FIG. 3.

Multiplexed analyses in shared fluid volumes may be used to conduct screens that measure potency and specificity of a condition, such as presence of a test compound, on a set of cell targets in a single screening step. In contrast, conventional high-throughput screens may be carried out with at least two distinct screens. Specifically, a first screen may identify conditions that show high potency on a desired target population of cells, and one or more additional screens may determine the specificity of the high potency conditions using other cell populations. Thus, conventional screens may require additional time, effort, and expense to identify compounds exhibiting high potency and specificity on cells, relative to a multiplexed approach performed in a shared fluid volume. Furthermore, a two-screen approach may overlook informative compounds that show lower potency but higher specificity. Such compounds may be important lead compounds in derivatization strategies that increase the potency of lead compounds by synthesizing and testing a set of lead-compound derivatives. In contrast to a conventional screening approach, multiplexed screens in shared fluid volumes may be well suited to identify lead compounds with lower potency but higher specificity.

FIG. 3 shows a graph 130 of results from a multiplexed screen that may be performed with coded carriers. The screen may measure the selectivity of a plurality of different conditions, using a set of cell populations as response targets in a shared fluid volume for each condition. The screen may be carried out in microplate wells (1-20), indicated at 132, with each well providing a shared fluid volume for exposure of the set of cell populations to a different condition. The cell populations may form a specificity panel, shown at 133. The specificity panel may include one or more desired targets 134, from which a selective response is desired. The specificity panel also may include one or more undesired targets 136, from which a weaker response or no response may be desired. In an exemplary embodiment, each cell population of the specificity panel may be engineered to express a different receptor. Accordingly, a response to a condition may be measured, for example, as activity of a reporter gene from each cell population. The reporter gene may be configured to be responsive to each different receptor, and may be the same or different for each cell population.

The cell populations also may provide a cytotoxicity panel 138. The cytotoxicity panel may include one or more additional cell populations from which a cytotoxicity signal or response may be measured. Alternatively, the cytotoxicity signal may be measured from one or more of the cell populations that form the specificity panel. The cytotoxicity signal may be a measure of apoptosis, necrosis, cell number, nuclear morphology, nuclear translocation of a cytotoxicity indicator (such as p53), activity of a stress response reporter, mitotic arrest, etc.

The cell populations may be used to perform a multiplexed assay as described above in relation to FIG. 1. Each cell population may be connected to a different class of one or more coded carriers and placed together in each of the wells. This placement may form a nonpositional array of cell populations in each well. The array of cells in each well may be exposed to a different condition and then a response, if any, of each cell population to the condition may be measured.

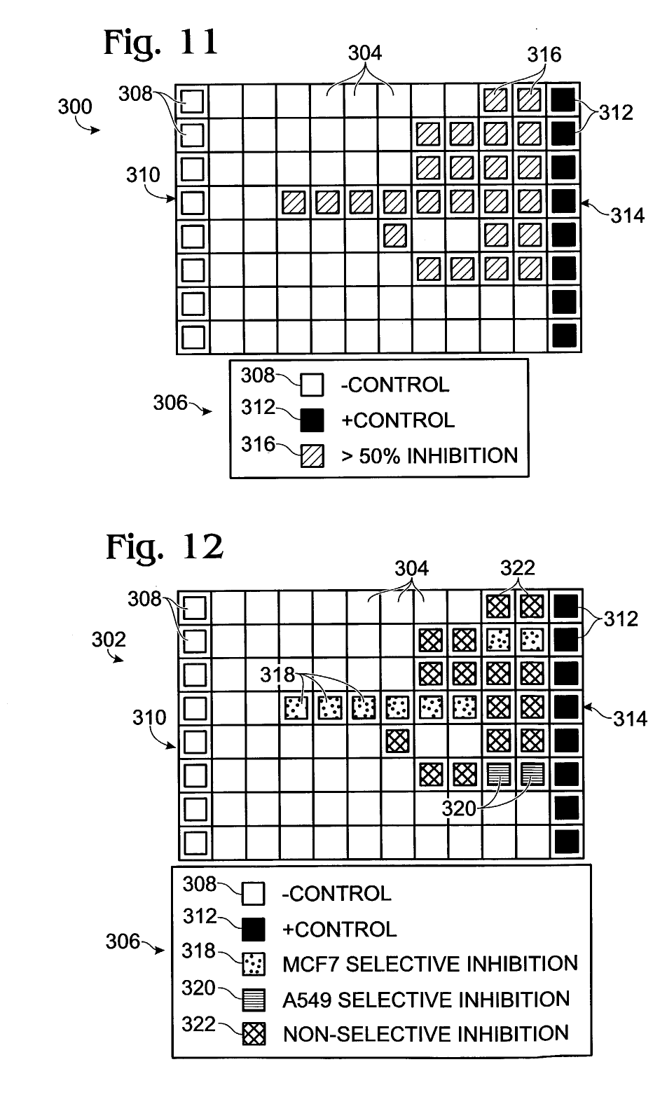

Graph 130 may display signals or responses measured from each of the cell populations in different shared fluid volumes. Signals 140 generated by exposure to a condition may be plotted by placing indicia, such as contours, bars, polyhedra, colors, shading, shapes, etc.), at a graphical array of sites defined by the wells (conditions) alone, or in combination with the cell populations (or by a parameter from each population). The indicia may be configured to mark the magnitude of each response and/or a selective effect (or lack thereof) on the responses produced by each condition. In some embodiments, the signals may result from measuring different parameters, and thus the magnitude and/or units may be different for the different parameters displayed within the graph. The wells (or conditions) may define a one-dimensional array of graphical sites on the graph, as shown here, so that each response generated from an assay in a shared fluid volume may be displayed separately, for example, in an array extending orthogonally to the one-dimensional array. Alternatively, the wells may define a two-dimensional array of graphical sites, and indicia may be selected and placed at the graphical sites according to selectivity of the condition for eliciting one or more of the responses (see FIGS. 11 and 12).

In the present illustration, potent responses 142, 144, 146, 148 on desired targets are displayed for four different wells holding cell populations exposed to four different conditions. However, three of these conditions, corresponding to responses 144, 146, 148, also have undesired nonspecific effects 150, corresponding to a substantial response from one or more of undesired targets 136, and/or cytotoxic effects 152, corresponding to a substantial response from cytotoxicity panel 138. Thus, this screen identifies a single condition, indicated by selective response 142, that produces a specific, nontoxic effect.

### EXAMPLE 2

Analysis of G-Protein Coupled Receptor Activity with Coded Carriers

This example describes various methods that may be used to measure G-Protein Coupled Receptor (GPCR) activity in cells connected to coded carriers; see FIG. 4.

A. Arrestin-Based Internalization Assays

An arrestin-based assay may be used to measure GPCR activation, generally irrespective of how the GPCR couples to G proteins and downstream signaling pathways. Arrestin is a cytoplasmic protein that associates with most activated (agonist-bound) GPCRs and promotes their clustering in coated pits and subsequent endocytosis. Accordingly, an optically detectable arrestin derivative, such an arrestin-GFP fusion protein, may be used to monitor GPCR activation and internalization as a result of agonist binding.

To test this approach, coded carriers were used to compare the subcellular distribution of arrestin-GFP before and after GPCR stimulation with agonist. Cells were engineered to express arrestin-GFP and connected to coded carriers. Without agonist stimulation, the fluorescent GFP signal was distributed fairly uniformly through the cytoplasm. By contrast, agonist stimulation of GPCR activity produced a GFP signal having a punctate pattern. At earlier time points after agonist addition, arrestin may be detected as recruited to the plasma membranes of the cells. Further aspects of the use of arrestin to measure GPCR activity are included in the following U.S. patents, which are incorporated herein by reference: U.S. Pat. No. 5,891,646, issued Apr. 6, 1999; and U.S. Pat. No. 6,110,693, issued Aug. 29, 2000. Further aspects of arrestin-based assays in cells connected to coded carriers are descried in U.S. Provisional Patent Application Serial No. 60/413,407, filed Sep. 24, 2002, which is incorporated herein by reference.

B. NF-κB-based Nuclear Translocation Assays

An NF-κB-based assay may be used to measure the activity of GPCRs that are coupled to G proteins of the Gα_{q} class. Activation of the NF-κB pathway results in degradation of IκB and translocation of NF-κB from the cytoplasm to the nucleus. Accordingly, a fusion of GFP to NF-κB, or an NF-κB-specific antibody, may be used to monitor activity of a GPCR that couples to the NF-κB pathway.

To test this approach, cells were engineered to express GFP-NF-κB and were connected to coded carriers. The cells were stimulated with a non-GPCR agonist, TNFα. NF-κB (GFP signal) was detected in the cytoplasm of cells prior to addition of the agonist TNFα, and then in the nucleus after addition of TNFα. More generally, the assay may be conducted with any agonist (or antagonist) for a GPCR that couples to the NF-κB pathway. Further aspects of NF-κB-based nuclear translocation assays in cells connected to coded carriers are described in U.S. Provisional Patent Application Serial No. 60/413,407, filed Sep. 24, 2002, which is incorporated herein by reference.

C. Calcium Assays

Multiplexed assays of GPCRs that couple to phospholipase Cβ may be performed with cell connected to coded carriers. GPCRs that couple to G proteins of the Gα_{q} class generally may be activated to increase phospholipase Cβ activity, producing increased levels of cytoplasmic calcium. The increased calcium levels may be produced by promoting uptake of extracellular calcium and/or by releasing calcium from intracellular storage sites. Calcium indicator dyes, such as fura-2 and fluo-3, may be used to measure the increased levels of intracellular calcium, by producing calcium-dependent changes in the optical properties of such dyes.

To test this approach, cell populations were connected to coded carriers and assayed for GPCR activity using a calcium indicator dye. Calcium signals were measured with cells expressing a bradykinin receptor, with or without agonist treatment. Cells without agonist exhibited a weak signal corresponding to low cytoplasmic calcium levels. Cells exposed to bradykinin exhibited a stronger fluorescent signal, corresponding to increased cytoplasmic calcium levels. Further aspects of calcium-based GPCR assays in cells connected to coded carriers are described in U.S. Provisional Patent Application Serial No. 60/413,407, filed Sep. 24, 2002, which is incorporated herein by reference.

D. cAMP Assays

GPCRs that couple to G proteins of the Gαₛ class may exhibit activity by stimulation of adenylyl cyclase activity, increasing levels of cyclic AMP (cAMP). These increased levels may be monitored using a cyclic-AMP-responsive reporter gene.

GPCR activity may be measured with a reporter gene assay. A cell expressing a GPCR protein may be incubated with a corresponding GPCR test ligand. The activity of the GPCR protein may be coupled to production of cAMP, for example, with Gα-coupled receptors. The cell may be modified to include a beta-galactosidase reporter gene controlled by a cAMP response element (CRE-βgal). Binding of ligand (agonist) to GPCR protein at the cell surface may activate the reporter gene, resulting in production of beta-galactosidase protein. Introducing a beta-galactosidase substrate, for example, a fluorogenic or chromogenic substrate, into the cell may produce a beta-galactosidase activity signal. This activity signal reports the ability of the test ligand to function as an agonist and acts as an indication of cell response.

To test this approach, increases in cAMP were measured with cells connected to coded carriers and expressing an adenylyl-cyclase-coupled GPCR. The cells were engineered to include a cAMP-responsive reporter gene. Response signals were measured before and after agonist addition. Agonist produced a marked increase in response signal.

FIG. 4 shows a graph 170 of comparative results obtained for activation of the diuretic hormone receptor in cells connected to microplate wells or to coded carriers. The cells were engineered to express the diuretic hormone receptor and a cAMP-responsive reporter gene that expresses beta-galactosidase. Beta-galactosidase activity 172 was plotted as a function of the diuretic hormone concentration 174 to which the cells were exposed. A range of concentrations of diuretic hormone was used to create dose response curves 176, 178, using microplate wells and coded carriers, respectively. Curves 176, 178 represent similar results, indicating the suitability of coded carriers for such assays. Further aspects of cAMP assays with cells connected to coded carriers are described in U.S. Provisional Patent Application Serial No. 60/413,407, filed Sep. 24, 2002, which is incorporated herein by reference.

### Example 3

Multiplexed Analysis of G Protein-Coupled Receptors

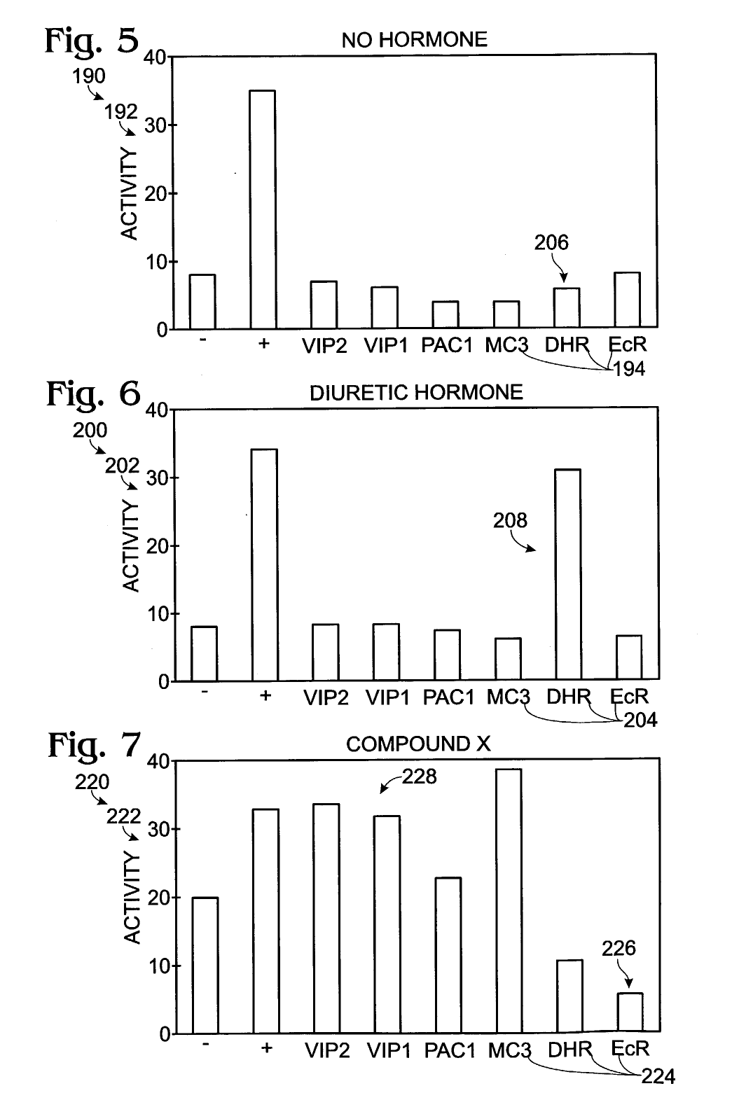

This example describes results obtained by multiplexed analysis of the activity of agonist and a test compound on a set of receptors carried in different cell populations connected to coded carriers; see FIGS. 5-7. The receptors include GPCRs, a nuclear hormone receptor, and positive/negative controls.

Agonist activity was measured in a multiplexed analysis with eight different cell populations or targets. The cell populations were modified or engineered by introduction of foreign expression vectors to express one of five distinct GPCRs (VIP1, VIP2, PAC1, MC3, or DHR), a Drosophila nuclear receptor (ecdysone receptor; EcR), or no exogenous receptor protein (controls). The GPCRs and the ecdysone receptor may signal through distinct signal transduction pathways and thus represent distinct mechanisms of action.

The GPCRs used in the assay couple to G proteins of the Gαₛ class, which stimulate cAMP production when activated by a GPCR. VIP1 and VIP2 are receptors for vasoactive intestinal polypeptide (VIP). Pancreatic acinar cells from most species express VIP receptors, and VIP is a potent stimulant of enzyme secretion. PAC 1 is a receptor for pituitary cyclase-activating polypeptide (PACAP). PACAP belongs to a large family of secretory peptides, including secretin, glucagon, VIP, and growth hormone releasing hormone. PACAP participates in a diverse array of physiological and developmental processes. MC3 is one of at least five melanocortin receptors. Melanocortin receptors respond to melanocortins, which are regulatory peptides produced by post-translational processing of pro-opiomelanocortin (POMC). Finally, DHR is an insect receptor for diuretic hormone.

Each of the cell populations also was engineered by transfection to include a suitable reporter gene. The cell populations that expressed foreign GPCR proteins and the cell population that acted as a negative (−) control were modified to carry a cAMP-responsive reporter gene that expressed beta-galactosidase (CRE-βgal). Consequently, beta-galactosidase enzyme activity reported GPCR activation in each of the GPCR-expressing cell populations, as described above. In contrast, the cell population that expressed ecdysone receptor carried an ecdysone-responsive control element regulating beta-galactosidase expression. Finally, the positive (+) control cell population carried a reporter gene that expressed beta-galactosidase constitutively from a viral promoter.

An array of target cell populations was created. Cells were transfected and connected to different classes of coded carriers. Each of the eight cell populations was generated by transfection of a substantially similar or clonal cell population using the GPCR/nuclear receptor expression vectors and/or reporter-gene nucleic acids described above. Then, each resultant, different cell population was connected separately to a class of coded carriers having a distinct code or set of codes by plating the cell population on a monolayer of the carriers. Alternatively, transfection may be carried out partially or completely by transfection of cells after they have been connected to distinct classes of carriers. The carrier classes and their connected cell populations were placed at arbitrary relative positions in each of a plurality of microplate wells. This placement provided a nonpositional array of coded cell populations in each well.

The arrays were assayed as follows. Each array was exposed to a condition (i.e., agonist, test compound, or control) in the wells of the microplate, with the position of each well/nonpositional array acting to identify each condition. After exposure to the condition, cell populations of each array were fixed, stained by incubation with the chromogenic beta-galactosidase substrate X-gal, and imaged. A beta-galactosidase signal was quantified after subtraction of a background signal from cell-free regions of the carriers. In some embodiments, a coding region(s) adjacent an assay region presents a code that identifies the cell population connected to each carrier and defines its position based on proximity to the coding region(s) and/or carrier. The assay region may be configured for measurement of the beta-galactosidase signal without significant optical interference from the code.

FIGS. 5 and 6 show graphs of results obtained from exposure of the cell populations to no agonist (as a control) and a known agonist, respectively. FIG. 5 shows a graph 190 of beta-galactosidase activity 192 measured from each cell population 194, without added hormone. FIG. 6 shows a graph 200 of beta-galactosidase activity 202 measured from each cell population 204 after exposure to diuretic hormone. Comparison of the two graphs shows that the cell population expressing the diuretic hormone receptor (DHR) exhibited an increase in beta-galactosidase activity in response to diuretic hormone exposure, shown by comparison of 206 and 208, but each other cell population showed no substantial change in activity. Accordingly, these results demonstrate that diuretic hormone is selective for DHR, relative to the five other receptors tested.

FIG. 7 shows a graph 220 of results obtained by testing each cell population after exposure to a test compound (“Compound X”). The test compound may be an agonist for an endogenous GPCR protein expressed in each of the cell populations. Beta-galatosidase activity 222 was plotted as a function of cell population 224 from which the activity was measured. Compound X does not affect ecdysone receptor (EcR) activity on an ecdysone-responsive reporter gene, shown at 226 (compare with FIG. 6), but produces significant activation of the CRE-βgal reporter gene in each cell population carrying this reporter gene, shown at 228. Accordingly, multiplexed assays in shared fluid volumes may provide appropriate controls in each shared fluid volume to determine 1) a response relative to a negative control, and/or 2) regulation of endogenous versus exogenous receptors. The controls also may allow different mechanisms of action of a condition to be distinguished.

### Example 4

Multiplexed Analyses of Receptor Responses

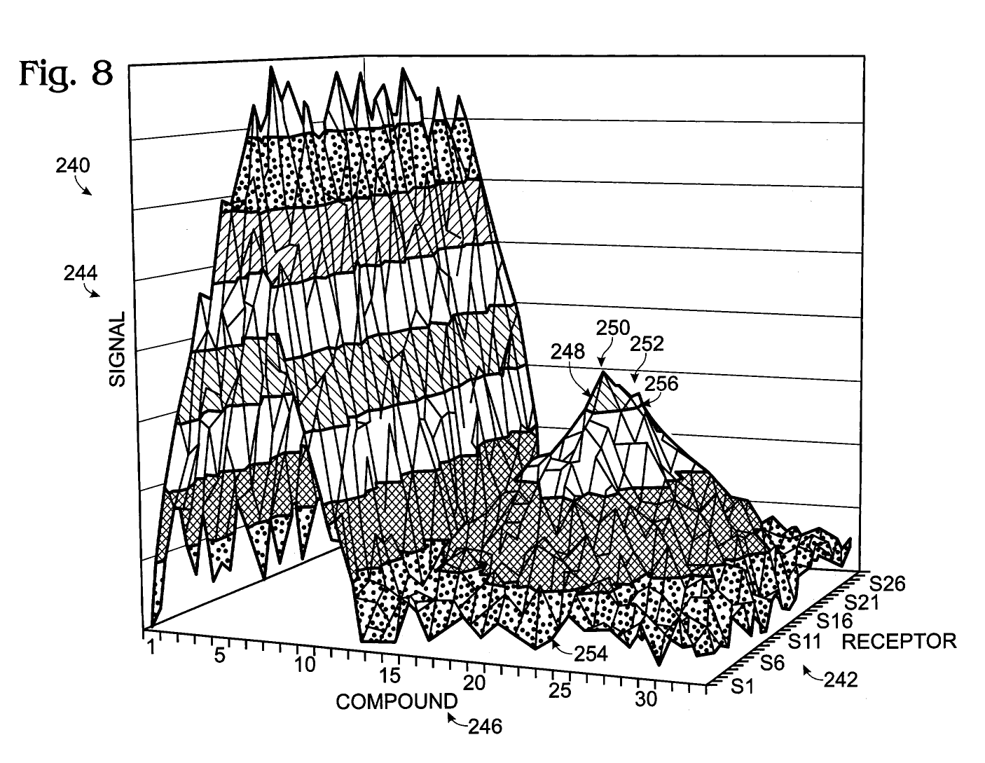

This example describes hypothetical results from multiplexed analyses performed using coded carriers to identify relationships between receptors/compounds and to define differences in potency and specificity of compounds, see FIG. 8.

Graph 240 displays data from screens of compounds for the ability to produce receptor activity. Cell populations expressing different receptors 242, indicated as S1-S26, are associated with different classes of coded carriers and combined in a nonpositional array for multiplexed analysis. Each cell population generates a measured signal 244, plotted along the vertical axis, in response to exposure to a compound 246. Signal 242 may be a reporter gene signal or other measured response of each cell population.

Graph 240 may display results from receptors 242 and test compounds 244 according to relatedness in structure or activity. Accordingly, receptors 242 that are placed near one another in graph 240 each may respond similarly to a positive test compound. For example, compound “23” elicits a similar signal from receptors plotted adjacent one another, shown at 248, 250, and 252. By contrast, a receptor plotted at a periphery of the receptor axis may generate a substantially weaker response with exposure to the same compound, shown at 254. Compounds also may be disposed in graph 240 according to structural or functional relatedness. Accordingly, adjacent compounds may elicit similar responses from a receptor, shown at 250 and 256.

### Example 5

Multiplexed Analysis of Cell Proliferation

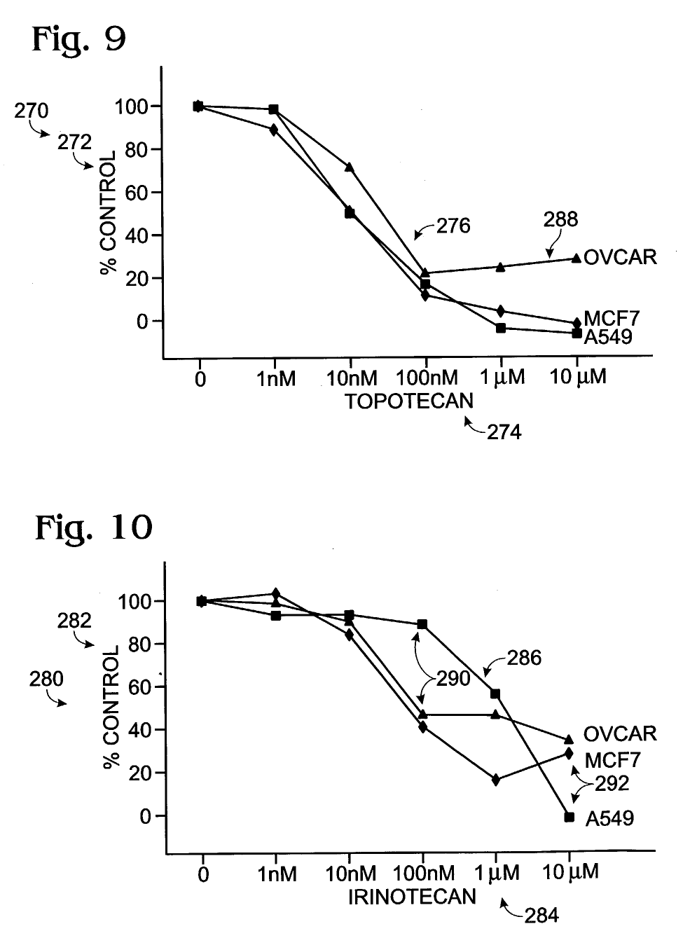

This example describes results of multiplexed analyses of cell proliferation performed using coded carriers, a plurality of different cell populations, and known and candidate proliferation inhibitors; see FIGS. 9-12.

Cell proliferation may be measured using any assay that provides a signal corresponding to mitosis. For example, mitosis may be reflected by a signal produced by cells in a particular phase of mitosis (G1, S, G2, or M), lack of a signal provided by quiescent cells (in G0), by increase in cell number, by nuclear size or morphology, etc.

In some embodiments, the signal may correspond to DNA synthesis, as an indication of cells progressing through S-phase. DNA synthesis may be measured by incorporation of a nucleotide analog, such a 5-bromo-2-deoxyuridine (BrdU), and subsequent detection of the analog, for example, after binding a dye-labeled partner, such as dye-labeled anti-BrdU antibody. Nuclear staining, such as with 7-AAD, may improve visualization of the nucleus when measuring the BrdU signal. Proliferation may be inhibited by inhibitors of DNA Topoisomerase I, which blocks DNA replication. Exemplary Topoisomerase I inhibitors include etoposide, camptothecin, topotecan, and irinotecan.

Multiplexed assays were performed with a set of different cell populations exposed in shared fluid volumes to different DNA Topoisomerase I inhibitors. Coded carriers of different classes were connected to different cell populations: OVCAR, MCF7, and A549. A mixture of the different classes and their connected cell populations were dispensed to each of a plurality of microplate wells, the cell populations were exposed to inhibitor for 48 hours. The cells were then fixed, incubated with FITC-conjugated anti-BrdU antibody for two hours, counterstained with 7-AAD, imaged to produce images, and the images analyzed to provide responses.

FIGS. 9 and 10 show results of exposure of the cell populations to different concentrations of topotecan and irinotecan, respectively. FIG. 9 shows a graph 270 in which a proliferation signal 272 relative to control is plotted according to topotecan concentrations 274 to which each cell population was exposed. Dose response curves 276 for the three types of cells are shown. FIG. 10 shows a graph 280 in which a proliferation signal 282 relative to control is plotted according to irinotecan concentrations 284 to which each cell population was exposed. Dose response curves 286 for the three types of cells are shown.

Comparison of dose-response curves 276 and 286 of FIGS. 9 and 10, respectively, indicate selective effects of each inhibitor on the different cell populations. For example, a selective effect of topotecan on MCF7 and A549 cells relative to OVCAR cells at high concentrations of the inhibitor is shown at 288. In addition, a selective effect of irinotecan on OVCAR and MCF7 cells relative to A549 cells is shown at 290 for 100 nM irinotecan. However, this effect is reversed at 10 μM irinotecan, shown at 292.

FIGS. 11 and 12 show results obtained from additional multiplexed assays of cell proliferation performed with coded carriers, a set of different cell types, and exposure to the cell types in shared fluid volumes to known and candidate cytotoxic compounds. Two different cell lines, MCF7 and A549, were connected to different classes of coded carriers. A mixture of the coded carriers and their connected cell types was placed in each well of a 96-well microplate. The first and last columns of microplate wells served as negative and positive controls, respectively. The cells contained in the other columns of wells were exposed in duplicate (adjacent pairs of wells in each row) to six known cytotoxic agents, or to members of a library of pharmaceutically active compounds (LOPAC) as candidate cytotoxic agents. The cells then were labeled with BrdU, fixed, stained with dye-labeled anti-BrdU antibody, imaged to collect images, and the images analyzed to measure responses. The responses correspond to a response of each cell population to exposure to a cytotoxic agent or control treatment, based on a measured BrdU signal.

FIGS. 11 and 12 show graphs 300 and 302, respectively, of potency and selectivity data obtained from these multiplexed assays. Each graph includes an array of sites 304 disposed in correspondence with examination sites or wells in which each multiplexed assay was conducted. Accordingly, each graph may include a two-dimensional array of sites 304. In alternative embodiments, each graph may include an array of sites corresponding to each condition (or a subset thereof) to which each array of cell populations was exposed, for example, as an average of experiments performed twice or more. Indicia 306 defining a potency and/or a selectivity may be placed at appropriate sites 304 in accordance with assay results. Such indicia may include symbols, colors, shapes, shading, stippling, hatching, text, and/or the like. For example, an open box 308 may be placed at sites 304 for which the corresponding wells served as negative controls, shown at 310. Here, the negative controls were produced by omitting BrdU from the assay. In addition, a solid box 312 may be placed at sites 304 for which the corresponding wells served as positive controls, shown at 314. Here, the positive controls were not exposed to a cytotoxic agent.

Indicia 306 of FIG. 11 may indicate potency of the cytotoxic agent. For example, hatched box 316 may indicate that one or more of the cytotoxic agents fall within a potency range or exceed (or do not exceed) a potency threshold for the response elicited from one or more of the cell populations. Here, box 316 indicates greater than 50% inhibition of one or more of the cell types. All sites 304 may include indicia or only a subset may include indicia.

Indicia 306 of FIG. 12 may indicate a selective effect of the cytotoxic agent on a subset of the cells types. The selective effect may be indicated for only the cytotoxic agents that show a predetermined inhibition potency (>50% in this case) or may be indicated for all wells. For example, stippled box 318 indicates an inhibition that is selective for MCF7 cells, that is, at least two fold greater for MCF7 than A549 cells. In addition, striped box 320 indicates an inhibition that is selective for A549 cells. Nonselectivity box 322 indicates a nonselective effect of the cytotoxic agent on the responses generated by the cells.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A method of multiplexed analysis of cells, comprising:
placing a set of isolated cell populations within a shared fluid volume and at least substantially segregated from one another;
exposing the cell populations to a condition;
measuring a response to the condition generated by each cell population to provide a set of responses; and
comparing the responses to define a selective effect, if any, of the condition.

2. The method of claim 1, wherein the step of placing a set of isolated cell populations disposes the cell populations at arbitrary positions relative to one another within the shared fluid volume.

3. The method of claim 1, wherein the step of placing a set of isolated cell populations includes placing a control cell population within the shared fluid volume, the control cell population generating a known response to the condition.

4. The method of claim 1, wherein the step of measuring a response includes identifying each cell population based on a relative or absolute position of the cell population within the shared fluid volume.

5. The method of claim 1, wherein the step of placing a set of isolated cell populations includes (1) connecting each cell population to a different class of one or more carriers, each different class having a code that identifies the different class, and (2) disposing each different class in the shared fluid volume.

6. The method of claim 1, wherein the step of placing a set of isolated cell populations includes (1) connecting the cell populations to separate positions of a microplate well in fluid isolation, and (2) creating fluid communication between the separate positions of the microplate well.

7. The method of claim 1, wherein each of the steps of placing, exposing, and measuring is performed at least partially in a well of a microplate.

8. The method of claim 7, further comprising repeating the steps of placing, exposing, and measuring in a plurality of wells of the microplate, the condition being different for each of the wells.

9. The method of claim 1, wherein the step of placing a set of isolated cell populations includes (1) selecting a set of similar cell populations, and (2) transfecting the similar cell populations with different transfection materials to create different cell populations.

10. The method of claim 9, wherein the step of transfecting is conducted in the shared fluid volume.

11. The method of claim 1, wherein the steps of placing and exposing are performed with cell populations having live cells, and wherein measuring a response includes measuring the response generated at least substantially by the live cells.

12. The method of claim 1, wherein the step of exposing the cell populations to a condition includes exposing the cell populations to a drug candidate.

13. The method of claim 1, wherein the step of measuring a response includes identifying each cell population based on a code associated with the cell population.

14. The method of claim 13, wherein the code is included in one or more coded carriers, and wherein the step of identifying each cell population includes 1) reading the code of at least one of the coded carriers, 2) locating a cell-association region defined by the at least one coded carrier, and 3) inferring position of the cell population based on proximity to the cell-association region.

15. The method of claim 14, wherein the step of measuring a response includes (1) obtaining at least one image of one or more different carrier classes, (2) analyzing the at least one image to identify the one or more different carrier classes, and (3) sensing a signal from each different class, the signal corresponding to the response generated by each cell population to the condition.

16. The method of claim 1, wherein the step of measuring a response includes (1) obtaining an image of a field of view, (2) identifying an area occupied by one of the cell populations within the image, (3) masking the image substantially complementary to the area, and (4) calculating a signal corresponding to the response from the area of the image after the step of masking.

17. The method of claim 16, wherein the step of identifying an area includes analyzing the image by reading one or more codes to determine the area.

18. The method of claim 1, wherein the step of measuring a response includes measuring the response quantitatively to define a potency of the condition on each cell population, and wherein the step of comparing includes comparing the potency of the condition on the different cell populations to define the selective effect.

19. The method of claim 1, wherein the step of measuring a response includes comparing a measured value to a control value or a predicted value to determine the response.

20. The method of claim 19, wherein the step of comparing a measured value includes measuring the control value in another fluid volume separate from the shared fluid volume.

21. The method of claim 19, wherein the step of comparing a measured value includes measuring the control value in the shared fluid volume.

22. The method of claim 1, wherein the cell populations include different receptors, and wherein the step of measuring a response includes detecting the response in relation to each of the different receptors.

23. The method of claim 22, wherein at least two of the different receptors belong to different classes that operate through different signal transduction pathways, and wherein the step of comparing includes comparing the response generated by exposure of each different receptor to the condition to define any impact of the condition on each of the different signal transduction pathways.

24. The method of claim 1, wherein at least a subset of the cell populations is engineered to include a reporter gene, and wherein measuring a response includes detecting a signal corresponding to expression of the reporter gene.

25. The method of claim 1, wherein the step of measuring a response includes measuring at least two different cell parameters for at least a subset of the cell populations.

26. The method of claim 1, wherein the step of measuring a response includes sensing a signal corresponding to at least one of the following: reporter gene activity, cytotoxicity, apoptosis, mitotic index, calcium flux, nuclear translocation, and DNA synthesis.

27. The method of claim 1, wherein the step of measuring a response includes determining whether a cell population is affected by the condition and, if so, how strongly it is affected.

28. A method of multiplexed analysis of cells, comprising:
placing a set of different cell populations in a shared fluid volume, each cell population being connected to a different class of one or more carriers, each class having a code that identifies the cell population connected to the class;
exposing each cell population to a condition in the shared fluid volume;
measuring a response to the condition generated by each cell population to provide a set of responses; and
comparing the responses to define a selective effect, if any, of the condition.

29. The method of claim 28, wherein the step of placing disposes the cell populations at arbitrary positions relative to one another within the shared fluid volume.

30. The method of claim 28, wherein the step of placing a set of different cell populations includes placing a control cell population within the shared fluid volume, the control cell population generating a known response to the condition.

31. The method of claim 28, wherein each of the steps of placing, exposing, and measuring is performed at least partially in a well of a microplate.

32. The method of claim 28, further comprising repeating the steps of placing, exposing, and measuring in a plurality of wells of the microplate, the condition being different for each of the wells.

33. The method of claim 28, wherein the step of placing a set of different cell populations includes (1) selecting a set of similar cell populations, and (2) transfecting the similar cell populations with different transfection materials to create the different cell populations.

34. The method of claim 33, wherein the step of transfecting is conducted in the shared fluid volume.

35. The method of claim 28, wherein the steps of placing and exposing are performed with cell populations having live cells, and wherein the step of measuring a response includes measuring the response generated at least substantially by the live cells.

36. The method of claim 28, wherein the step of exposing each cell population to a condition includes exposing the cell population to a drug candidate.

37. The method of claim 28, wherein the step of measuring a response includes identifying each cell population based on the code of the class to which the cell population is connected.

38. The method of claim 37, wherein the step of identifying each cell population includes 1) reading the code of the class of carrier to which the cell population is connected, 2) locating a cell-association region defined by such class, and 3) inferring position of the cell population based on proximity to the cell-association region.

39. The method of claim 28, wherein the step of measuring a response includes (1) obtaining an image of a field of view, (2) identifying an area occupied by one of the cell populations within the image, (3) masking the image substantially complementary to the area, and (4) calculating a signal corresponding to the response from the area of the image after the step of masking.

40. The method of claim 39, wherein the step of identifying an area includes analyzing the image by reading one or more codes to determine the area.

41. The method of claim 28, wherein the step of measuring a response includes measuring the response quantitatively to define a potency of the condition on each cell population, and wherein the step of comparing includes comparing the potency of the condition on the different cell populations to define the selective effect.

42. The method of claim 28, wherein the step of measuring a response includes comparing a measured value to a control value or a predicted value to determine the response.

43. The method of claim 42, wherein the step of comparing a measured value includes measuring the control value in another fluid volume separate from the shared fluid volume.

44. The method of claim 42, wherein the step of comparing a measured value includes measuring the control value in the shared fluid volume.

45. The method of claim 28, wherein the cell populations include different receptors, and wherein the step of measuring a response includes detecting the response in relation to each of the different receptors.

46. The method of claim 45, wherein at least two of the different receptors belong to different classes that operate through different signal transduction pathways, and wherein the step of comparing includes comparing the response generated by exposure of each different receptor to the condition to define any impact of the condition on each of the different signal transduction pathways.

47. The method of claim 28, wherein at least a subset of the different cell populations is engineered to include a reporter gene, and wherein measuring a response includes detecting a signal corresponding to expression of the reporter gene.

48. The method of claim 28, wherein the step of measuring a response includes measuring at least two different cell parameters for at least a subset of the cell populations.

49. The method of claim 28, wherein the step of measuring a response includes determining whether a cell population is affected by the condition and, if so, how strongly it is affected.

50. A system for multiplexed analysis of cells, comprising:
a vessel defining a fluid volume;
two or more cell populations disposed in a segregated configuration in the vessel and in fluid communication within the fluid volume;
an imaging device configured to acquire at least one image of the cell populations, the at least one image including identifying information and response information for each cell population; and
an image analysis device that uses the identifying information and the response information from the at least one image to identify each cell population, to determine a response to exposure to a condition for each cell population and thereby provide a set of responses, and to compare the responses to define a selective effect, if any, of the condition.

51. The system of claim 50, wherein the vessel is a microplate well.

52. The system of claim 51, wherein the microplate well is subdivided into a plurality of sub-wells, the sub-wells being configured to hold the cell populations in fluid isolation, if desired.

53. The system of claim 50, wherein the vessel is a plurality of microplate wells, each well including a different test compound and holding the cell populations in a segregated configuration and in fluid communication with one another, and wherein the image analysis device is configured to define the selective effect of each test compound.

54. The system of claim 50, further comprising a set of carriers of at least two classes, each class having a different code and being connected to a different cell population.

55. The system of claim 54, wherein the identifying information includes code information corresponding to each different code.

56. The system of claim 50, wherein the condition is presence of a test compound.

57. The system of claim 50, wherein the cell populations include similar cell populations engineered to be different.

58. The system of claim 50, wherein the two or more cell populations include three or more cell populations.

59. The system of claim 50, wherein the vessel includes a surface, and wherein each of the cell populations is connected to the vessel adjacent the surface.

60. The system of claim 50, the response relating to at least one of: cell proliferation, DNA replication, mitotic index, cytoxicity, reporter gene expression, and calcium flux.

61. The system of claim 50, wherein the image analysis device is configured to determine a signal corresponding to the response of a cell population by masking the image to block a subset of the response information generated by other cell populations.

62. A database of data corresponding to a set of responses generated by exposure of two or more different cell populations to a plurality of different conditions, the database being obtained using the method of claim 1.

63. A database of data corresponding to a set of responses generated by exposure of two or more different cell populations to a plurality of different conditions, the database being obtained using the method of claim 28.

64. A database of data corresponding to a set responses generated by exposure of two or more different cell populations to a plurality of different conditions, the database being obtained using the system of claim 50.

65. A method of presenting data obtained by multiplexed assay of responses generated by two or more cell populations with exposure of the cell populations in a shared volume to a plurality of different conditions, comprising:
creating a graphical array of sites corresponding to the plurality of different conditions;
selecting indicia that represent the responses produced with exposure to each condition; and
placing the indicia at the sites in correspondence with the different conditions that produced the responses represented by the indicia.

66. The method of claim 65, wherein the graphical array of sites is a one-dimensional array.

67. The method of claim 66, wherein the indicia are configured to mark magnitude of the responses, and wherein the step of placing positions the indicia along an axis that is at least substantially orthogonal to the one-dimensional array.

68. The method of claim 65, wherein the graphical array of sites is a two-dimensional array.

69. The method of claim 65, wherein the step of selecting indicia includes selecting selectivity indicia representing a selective effect of a condition on a subset of the responses.

70. The method of claim 69, wherein the step of selecting selectivity indicia includes selecting one or more indicia indicating lack of a selective effect produced by a condition.

71. The method of claim 65, wherein the step of selecting indicia includes selecting different colors to represent different selective effects of a condition on the responses generated by the two or more cell populations.

72. The method of claim 65, wherein the step of placing is performed with a digital computing device.
